(19) United States
(12) Patent Application Publication
(10) Pub. No.: US 2017/0313851 A1
(43) Pub. Date: Nov. 2, 2017

(54) ESTER PLASTICIZERS BASED ON ETHYLHEXANOL AND PROPYLHEPTANOL

(71) Applicant: EMERY OLEOCHEMICALS GMBH, Düsseldorf (DE)

(72) Inventors: Peter Daute, Beverstedt (DE); Martin Schaefer, Stubben (DE)

(21) Appl. No.: 15/523,509

(22) PCT Filed: Dec. 2, 2014
(86) PCT No.: PCT/EP2014/076288

§ 371 Date: May 1, 2017

(51) Int. Cl. C08K 5/12; C07C 69/76; C07C 67/12

(52) U.S. Cl. CPC C08K 5/12; C07C 67/12; C07C 69/76

(57) ABSTRACT

The present invention relates to an ester obtainable by reacting (a) an alcohol mixture comprising 20-50 wt % ethylhexanol, 40-64 wt % propylheptanol and 0-20 wt % of other alcohols with (b) a polycarboxylic acid. Further aspects of the invention relate to a thermoplastic polymer comprising the ester according to the invention and uses of said ester.

The present invention relates to an ester obtainable by reacting (a) an alcohol mixture comprising 20-50 wt % ethylhexanol, 40-64 wt % propylheptanol and 0-20 wt % of other alcohols with (b) a polycarboxylic acid. Further aspects of the invention relate to a thermoplastic polymer comprising the ester according to the invention and uses of said ester.

### BACKGROUND OF THE INVENTION

Plasticizers are additives that increase the flexibility, pliability, and plasticity or fluidity of a material. The main applications are for plastics, especially thermoplastic compositions. Plasticizers are useful for a variety of polymers and applications such as wire-and-cable insulations, wire-and-cable jackets, coatings, adhesives, and castings—just to name some applications.

In order to change the mechanical properties of material plasticizers can be admixed. Thereby, flexible and plastic compounds can be obtained. For example, to the thermoplastic polymer polyvinyl chloride (PVC) a plasticizer can be added in such a large amount that PVC can be processed as a paste (see Gächter/Müller, Kunstoffadditive, 2. Auflage Carl Hanser Verlag).

For applications that need to resist higher temperatures plasticizers such as phthalates and aromatic tricarboxylic esters including trimellitic acid esters can be used. Known in the prior art is for example the compound tri-2-ethylhexyl trimellitate. One disadvantage of this compound, however, is that it is still volatile. Thus, the utility of this plasticizer is reduced in particular for applications that need to resist higher temperatures, since at such temperatures the plasticizer will evaporate from the thermoplastic polymer. However, the permanence over time of a plasticizer in the polymer, even when exposed to relatively high temperatures is an important requirement for many technical applications involving plastic compositions.

With respect to phthalates prior art plasticizers include for example di(ethyl hexyl)phthalate (“DEHP”), di-isononyl phthalate (“DINP”), and other phthalate plasticizers. However, phthalate plasticizers are in general less frequently used since this plasticizer type is linked to concerns over potential adverse health effects.

Plasticizers are particularly useful if they have a reduced volatility at higher temperatures but have at the same time also a low pour point. A low pour point ensures that the final product comprising the plasticizer remains flexible even at low temperatures. A reduced volatility at higher temperatures will also preserve the plasticizer's effect in the product also at high temperatures as is required for example for thermoplastic polymer compositions used for the electrical insulation of automotive electrical components. Thus, a plasticizer having the aforementioned properties will increase the versatility of its use.

Thus, there is a general need in the art for novel plasticizers that do not have the above outlined disadvantages.

### SUMMARY OF THE INVENTION

The present invention provides in a first aspect an ester obtainable by reacting

(a) an alcohol mixture comprising
(i) 20-50 wt % ethylhexanol, based on the total weight of the alcohol mixture
(ii) 40-64 wt % propylheptanol, based on the total weight of the alcohol mixture
(iii) 0-20 wt % other alcohols, based on the total weight of the alcohol mixture

with

(b) a polycarboxylic acid or anhydride thereof.

Preferably said ethylhexanol is 2-ethylhexanol and said other alcohols are a decanol that is different from 2-propylheptanol or an alcohol mixture comprising such decanols. Also provided is a thermoplastic polymer comprising the ester according to the invention.

A further aspect of the invention relates to the use of the ester of the invention as plasticizer in a thermoplastic polymer.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Some documents are cited throughout the text of this specification. Each of the documents cited herein (including all patents, patent applications, scientific publications, manufacturer's specifications, instructions, DIN norms etc.), whether supra or infra, are hereby incorporated by reference in their entirety. Nothing herein is to be construed as an admission that the invention is not entitled to antedate such disclosure by virtue of prior invention.

Novel plasticizers were identified that have a reduced volatility at higher temperatures and are also characterized by a low pour point. Accordingly, the invention provides in a first aspect an ester obtainable by reacting

(a) an alcohol mixture comprising
(i) 20-50 wt % ethylhexanol, based on the total weight of the alcohol mixture
(ii) 40-64 wt % propylheptanol, based on the total weight of the alcohol mixture
(iii) 0-20 wt % other alcohols, based on the total weight of the alcohol mixture

with

(b) a polycarboxylic acid or anhydride thereof.

Preferably said polycarboxylic acid used to make the ester is an aromatic polycarboxylic acid and more preferably said polycarboxylic acid is an aromatic tricarboxylic acid.

In a further preferred embodiment the ester according to the invention is a triester. For example, the ester of the invention may be a triester obtainable by reacting

(a) an alcohol mixture comprising
(i) 20-50 wt % ethylhexanol, based on the total weight of the alcohol mixture
(ii) 40-64 wt % propylheptanol, based on the total weight of the alcohol mixture
(iii) 0-20 wt % other alcohols, based on the total weight of the alcohol mixture

with

(b) an aromatic tricarboxylic acid or anhydride thereof.

Preferably, components (i), (ii) and (iii) together are in total 100 wt % of the alcohol mixture, i.e. the alcohol mixture (a) of the invention preferably consists of components (i), (ii) and (iii). In a preferred embodiment of the ester, said polycarboxylic acid is selected from the group consisting of mellitic acid, trimellitic acid, pyromelitic acid, hemimellitic acid, trimesic acid, naphthalenetricarboxylic acid, anthracenetricarboxylic acid, biphenyltricarboxylic acid, benzophenonetricarboxylic acid and mixtures and position isomers of the aforementioned acids.

In a further preferred embodiment, the alcohol mixture consists of

(i) 20-50 wt % ethylhexanol, based on the total weight of the alcohol mixture; and

(ii) 40-64 wt % propylheptanol, based on the total weight of the alcohol mixture; and

(iii) 0-20 wt % other alcohols, based on the total weight of the alcohol mixture.

“Other alcohols” as used herein are preferably alcohols other than those used as components (i) and (ii). In a more preferred embodiment, “other alcohols” are branched primary alcohols and even more preferably a decanol other than 2-propylheptanol or an alcohol mixture comprising such decanols.

Most preferably, “other alcohols” refers to a branched primary decanol different from 2-propylheptanol or refers to an alcohol mixture consisting of two or more of such branched primary decanols.

In a further preferred embodiment of the ester according to the invention the alcohol mixture comprises

30-45 wt % ethylhexanol, based on the total weight of the alcohol mixture; and

the remaining alcohols in the alcohol mixture consist of propylheptanol and other alcohols,

wherein said other alcohols are not more than maximally 20 wt % of the total weight of the alcohol mixture.

It is further preferred that the alcohol mixture comprises

30-45 wt % ethylhexanol, based on the total weight of the alcohol mixture; and

the remaining alcohols in the alcohol mixture consist of propylheptanol and other alcohols,

wherein said other alcohols are not more than maximally 15 wt % of the total weight of the alcohol mixture.

In a more preferred embodiment the alcohol mixture comprises

30-35 wt % ethylhexanol, based on the total weight of the alcohol mixture; and

the remaining alcohols in the alcohol mixture consist of propylheptanol and other alcohols,

wherein said other alcohols are not more than maximally 15 wt % of the total weight of the alcohol mixture.

In a further preferred embodiment, the alcohol mixture comprises

38-50 wt % ethylhexanol, based on the total weight of the alcohol mixture; and

the remaining alcohols in the alcohol mixture consist of propylheptanol and other alcohols,

wherein said other alcohols are not more than maximally 15 wt % of the total weight of the alcohol mixture.

In a more preferred embodiment the alcohol mixture comprises

40-45 wt % ethylhexanol, based on the total weight of the alcohol mixture; and

the remaining alcohols in the alcohol mixture consist of propylheptanol and other alcohols,

wherein said other alcohols are not more than maximally 12 wt % of the total weight of the alcohol mixture.

Alternatively, the ester of the invention may also be based on an alcohol mixture that comprises

20-37 wt % ethylhexanol, based on the total weight of the alcohol mixture; and

the remaining alcohols in the alcohol mixture consist of propylheptanol and 5 other alcohols,

wherein said other alcohols are not more than maximally 16 wt % of the total weight of the alcohol mixture.

In a particularly preferred embodiment of the ester according to the invention said ethylhexanol is 2-ethylhexanol and said propylheptanol is 2-propylheptanol.

A further aspect of the invention relates to a thermoplastic polymer comprising the ester according to the invention.

Thereby it is preferred that the thermoplastic polymer is selected from the group consisting of polyethylene, PET, PC, ABS, polyamide, TPU, TPE, nitrile butadiene rubber (NBR), hydrogenated nitrile butadiene rubber (HNBR), chloroprene rubber (CR), polystyrene and a polyolefin, preferably a halogenated polyolefin such as polyvinyl chloride.

Preferably, the thermoplastic polymer is a halogenated polymer. More preferably, the halogenated polymer is a PVC polymer selected from the group consisting of PVC homopolymers, PVC copolymers, polyvinyl dichlorides (PVDC), and polymers of vinylchloride with vinyl, acrylic and other co-monomers.

In a further aspect the invention provides the use of the ester of the invention as defined herein as plasticizer in a thermoplastic polymer.

A preferred embodiment of the use according to the invention relates to a use, wherein the thermoplastic polymer is selected from the group consisting of polyethylene, PET, PC, ABS, polyamide, TPU, TPE, nitrile butadiene rubber (NBR), hydrogenated nitrile butadiene rubber (HNBR), chloroprene rubber (CR), polystyrene and a polyolefin, preferably a halogenated polyolefin such as polyvinyl chloride.

Various modifications and variations of the invention will be apparent to those skilled in the art without departing from the scope of the invention. Although the invention has been described in connection with specific preferred embodiments, it should be understood that the invention as claimed should not be unduly limited to such specific embodiments.

The following examples are merely illustrative of the present invention and should not be construed to limit the scope of the invention as indicated by the appended claims in any way.

### EXAMPLES

### Example 1: Ester Production

The amount of 2-ethylhexanol (“2 EH”) and 2-propylheptanol (“2 PH”) indicated in table 1 below was heated under nitrogen with trimellitic acid anhydride (“TMA”; respective amounts see table) and 500 ppm of tin-(II)-oxalate (Evonic), based on the total amount of the composition. The 2-propylheptanol that was used was of technical grade purity and therefore consisted of 80% 2-propylheptanol and 20% decanol isomers other than 2-propylheptanol. The esterification reaction begins at about 150° C. with the formation of water and the temperature is slowly increased to 220° C. After 4 hours the reaction water was further removed and the vacuum was reduced over 10 hours to about 5 mbar. The reaction was stopped when the acid number dropped below 1. Remaining unreacted alcohol was removed by distillation. Thereafter, the product was filtered.

TABLE 1
Amounts of compounds used

| Example | 2 EH in g | 2 PH in g | TMA in g | Yield in g |
|---|---|---|---|---|
| 1 | 996 | 0 | 352 | 998 |
| 2 | 682 | 415 | 403 | 1163 |
| 3 | 990 | 1370 | 837 | 2430 |
| 4 | 343 | 755 | 406 | 1228 |
| 5 | 0 | 1134 | 367 | 1153 |

### Example 2: Determination of Physical Properties of the Ester

The degree of discoloration of the ester was measured in Hazen units according to the method described in DIN EN ISO 6271-2005.

The acid number was determined according to DIN 53402.

The viscosity was measured using DIN 53019.

Furthermore, the pourpoint, which indicates the lowest temperature at which a liquid becomes semi solid and loses its flow characteristics, was determined according to DIN ISO 3016(10/82).

The cloud point was determined according 5 to DIN EU 23015.

Volatility of the ester was tested using a Netzsch instrument for thermogravimetry by heating a sample under N2 from 20° C. to a final temperature of 300° C. at a rate of at a 10 K/min. After the sample reached a temperature of 300° C. the sample was kept at this temperature for 30 minutes.

The results are shown in table 2 and 3 below:

TABLE 2

| | | Acid number | Calculation | Viscosity 20° C. |
|---|---|---|---|---|
| Example | Color Hazen | (mg KOH/g) | index 20° C. | (mPa · s) |
| 1 | 95 | 0.2 | 1.4853 | 300 |
| 2 | 96 | 0.09 | 1.4844 | 333 |
| 3 | 88 | 0.12 | 1.4837 | 364 |
| 4 | 108 | 0.11 | 1.4835 | 368 |
| 5 | 159 | 0.74 | 1.4826 | 401 |

TABLE 3

| | | % weight loss of ester | |
|---|---|---|---|
| | | after heat treatment as | |
| Example | pour-point | described | cloud point |
| 1 | −42 | 93% | −25 |
| 2 | −36 | n.d. | n.d. |
| 3 | −40 | 70% | −35 |
| 4 | −45 | n.d. | n.d. |
| 5 | −35 | 56% | −17 |

Examples 3 and 4 are according to the invention

### Example 3: Thermoplastic Composition Comprising Ester

The following compositions were prepared:

TABLE 4
Preparation of test specimens

| | Example: | | | | |
|---|---|---|---|---|---|
| | B6 | B7 | B8 | B9 | B10 |
| Polyvinyl chloride | 100 | 100 | 100 | 100 | 100 |
| Edenol D 81 | 3 | 3 | 3 | 3 | 3 |
| (epoxidized soybean oil) | | | | | |
| Stabiol CZ 2222 | 2 | 2 | 2 | 2 | 2 |
| (Ca/Zn-based stabilizer) | | | | | |
| Ester of example 1 | 40 | — | — | — | — |
| Ester of example 2 | — | 40 | — | — | — |
| Ester of example 3 | — | — | 40 | — | — |
| Ester of example 4 | — | — | — | 40 | — |
| Ester of example 5 | — | — | — | — | 40 |

The components as shown above were mixed together and milled on a laboratory rolling mill for 5 minutes at 185° C. Using rolled sheets 3 mm press plates were produced in a laboratory press at 180° C. and on these test specimens were examined according to DIN ISO 868 for their Shore A hardness (three measurements per sample). The results are outlined in table 5 below:

| | Example: | | | | |
|---|---|---|---|---|---|
| | B6 | B7 | B8 | B9 | B10 |
| Shore A | 94.8 | 96.9 | 96.9 | 97.6 | 97.8 |
| | 96.3 | 96.6 | 96.9 | 98.11 | 97.7 |
| | 95.1 | 96.6 | 96.9 | 98.0 | 97.7 |

Examples B8 and B9 are according to the invention.

As can be seen from the examples above, the optimized ester of the invention can be used as plasticizer in the processing of thermoplastic resins. The ester shows good compatibility with the polymer, low volatilities and also provides excellent low temperature properties.

1. An ester obtainable by reacting
(a) an alcohol mixture comprising
(i) from 20-50 wt % ethylhexanol, based on the total weight of the alcohol mixture
(ii) from 40-64 wt % propylheptanol, based on the total weight of the alcohol mixture
(iii) from 0-20 wt % other alcohols, based on the total weight of the alcohol mixture
with
(b) a polycarboxylic acid or anhydride thereof

2. The ester according to claim 1, wherein the polycarboxylic acid is an aromatic polycarboxylic acid.

3. The ester according to claim 2, wherein the aromatic polycarboxylic acid is an aromatic tricarboxylic acid.

4. The ester according to claim 1, wherein the ester is a triester.

5. The ester according to claim 1, wherein the polycarboxylic acid is selected from the group consisting of mellitic acid, trimellitic acid, pyromelitic acid, hemimellitic acid, trimesic acid, naphthalenetricarboxylic acid, anthracenetricarboxylic acid, biphenyltricarboxylic acid, benzophenonetricarboxylic acid and mixtures and position isomers of the aforementioned acids.

6. The ester according to claim 1, wherein the alcohol mixture consists of
(i) from 20-50 wt % ethylhexanol, based on the total weight of the alcohol mixture; and
(ii) from 40-64 wt % propylheptanol, based on the total weight of the alcohol mixture;
and
(iii) from 0-20 wt % other alcohols, based on the total weight of the alcohol mixture.

7. The ester according to claim 1, wherein the alcohol mixture comprises 30-45 wt % ethylhexanol, based on the total weight of the alcohol mixture; and the remaining alcohols in the alcohol mixture consist of propylheptanol and other alcohols, wherein said other alcohols are not more than maximally 20 wt % of the total weight of the alcohol mixture.

8. The ester according to claim 1, wherein the alcohol mixture comprises 30-45 wt % ethylhexanol, based on the total weight of the alcohol mixture; and the remaining alcohols in the alcohol mixture consist of propylheptanol and other alcohols, wherein said other alcohols are not more than maximally 15 wt % of the total weight of the alcohol mixture.

9. The ester according to claim 1, wherein the alcohol mixture comprises 38-50 wt % ethylhexanol, based on the total weight of the alcohol mixture; and the remaining alcohols in the alcohol mixture consist of propylheptanol and other alcohols, wherein said other alcohols are not more than maximally 15 wt % of the total weight of the alcohol mixture.

10. The ester according to claim 1, wherein the alcohol mixture comprises 20-37 wt % ethylhexanol, based on the total weight of the alcohol mixture; and the remaining alcohols in the alcohol mixture consist of propylheptanol and other alcohols, wherein said other alcohols are not more than maximally 16 wt % of the total weight of the alcohol mixture.

11. The ester according to claim 1, wherein ethylhexanol is 2-ethylhexanol and propylheptanol is 2-propylheptanol.

12. A thermoplastic polymer comprising the ester according to claim 1.

13. The thermoplastic polymer according to claim 12, wherein the thermoplastic polymer is selected from the group consisting of polyethylene, PET, PC, ABS, polyamide, TPU, TPE, nitrile butadiene rubber (NBR), hydrogenated nitrile butadiene rubber (HNBR), chloroprene rubber (CR), polystyrene and a polyolefin, preferably a halogenated polyolefin such as polyvinyl chloride.

14. A thermoplastic polymer comprising the ester according to claim 2.

15. The thermoplastic polymer according to claim 14, wherein the thermoplastic polymer is selected from the group consisting of polyethylene, PET, PC, ABS, polyamide, TPU, TPE, nitrile butadiene rubber (NBR), hydrogenated nitrile butadiene rubber (HNBR), chloroprene rubber (CR), polystyrene and a polyolefin, preferably a halogenated polyolefin such as polyvinyl chloride.

16. The ester according to claim 2, wherein the alcohol mixture comprises 30-45 wt % ethylhexanol, based on the total weight of the alcohol mixture; and the remaining alcohols in the alcohol mixture consist of propylheptanol and other alcohols, wherein said other alcohols are not more than maximally 20 wt % of the total weight of the alcohol mixture.

17. The ester according to claim 2, wherein the alcohol mixture comprises 30-45 wt % ethylhexanol, based on the total weight of the alcohol mixture; and the remaining alcohols in the alcohol mixture consist of propylheptanol and other alcohols, wherein said other alcohols are not more than maximally 15 wt % of the total weight of the alcohol mixture.

18. The ester according to claim 2, wherein the alcohol mixture comprises 38-50 wt % ethylhexanol, based on the total weight of the alcohol mixture; and the remaining alcohols in the alcohol mixture consist of propylheptanol and other alcohols, wherein said other alcohols are not more than maximally 15 wt % of the total weight of the alcohol mixture.

19. The ester according to claim 2, wherein the alcohol mixture comprises 20-37 wt % ethylhexanol, based on the total weight of the alcohol mixture; and the remaining alcohols in the alcohol mixture consist of propylheptanol and other alcohols, wherein said other alcohols are not more than maximally 16 wt % of the total weight of the alcohol mixture.

20. The ester according to claim 2, wherein ethylhexanol is 2-ethylhexanol and propylheptanol is 2-propylheptanol.
